(19) United States
(12) Patent Application Publication
(10) Pub. No.: US 2014/0336946 A1
(43) Pub. Date: Nov. 13, 2014

(54) METHOD AND SYSTEM FOR OBTAINING CYCLE OF PHYSIOLOGICAL SIGNAL

(71) Applicant: SHENZHEN SEELEN TECHNOLOGY CO., LTD., Shenzhen, Guangdong (CN)

(72) Inventors: Xufang Liu, Shenzhen (CN); Song Yang, Shenzhen (CN)

(21) Appl. No.: 14/362,414

(22) PCT Filed: Feb. 1, 2013
(86) PCT No.: PCT/CN2013/071291

§ 371 Date: Jun. 3, 2014

(30) Foreign Application Priority Data
Mar. 12, 2012 (CN) 201210063536.9

(51) Int. Cl. A61B 5/00

(52) U.S. Cl. CPC A61B 5/7271
USPC 702/19

(57) ABSTRACT

The present disclosure provides a method and a system for obtaining a cycle of a physiological signal. The method includes: receiving a physiological signal value and a register value, comparing the physiological signal value with the register value, and reserving one of the physiological signal value and the register value; determining the physiological signal value with a time duration thereof reaching a given set time to be an extreme value; and restarting the procedure and determining a next extreme value; and obtaining a cycle of the physiological signal by calculating a time difference between the extreme value and the next extreme value. In the present disclosure, the cycle of the physiological signal can be obtained by the extreme value recognition algorithm, which is simple, fast, of high efficiency, and of high reliability.

### BACKGROUND

1. Technical Field

The present disclosure generally relates to technologies for obtaining physiological signals, and more particularly, to a method and a system for obtaining a cycle of a physiological signal.

2. Description of Related Art

Important physiological signals of a human body, such as heartbeat signals and respiration signals, were always obtained by collecting and processing electromyography signals. A signal collecting device needed to contact in tight with human skin for acquiring clear signals which are thereafter amplified and processed. A cycle of the physiological signal obtained in the above way can be obtained as follows: a simple threshold at first is used to set a shaping and obtain a flag bit related to the cycle, then the cycle is obtained by calculating the flag bit of the cycle.

A micro-motion signal of a human body such as a heartbeat signal or a respiration signal (as shown in FIG. 1) sensed by a micro-motion sensor, has a waveform which is totally different from that of a bioelectric signal (as shown in FIG. 2). The waveform includes a different number of wave beams in a skip cycle, a magnitude of the waveform changes linearly according to the cycle, and the cycle cannot be calculated by threshold shaping or simple Fourier transformation. At present, the most common method to calculate the cycle is the real-time heartbeat rate recognition. However, the real-time heartbeat rate recognition method uses the auto-correlation function with a large amount of computation, making it hard to operate the real-time heartbeat rate recognition method in cheap ARMs and thus increasing the cost of obtaining the cycle.

### SUMMARY

The main object of the present disclosure is to provide a method for obtaining a cycle of a physiological signal which improves the efficiency of obtaining the cycle of the physiological signal and reduces the cost of obtaining the cycle.

The method for obtaining a cycle of a physiological signal provided in the present disclosure includes:
receiving a physiological signal value and a register value, comparing the physiological signal value with the register value, and reserving one of the physiological signal value and the register value; determining the physiological signal value with a time duration thereof reaching a given set time to be an extreme value; and restarting the procedure and determining a next extreme value; and
obtaining a cycle of the physiological signal by calculating a time difference between the extreme value and the next extreme value.

Preferably, the method further includes the following steps:
judging whether two cycles respectively obtained in a maximum value way and in a minimum value way in a time duration are close to each other; and
determining an average of the two cycles to be the cycle of the physiological signal if the two cycles are close to each other.

Preferably, the step of receiving a physiological signal value and register value and comparing the physiological signal value with the register value; determining the physiological signal value with a time duration thereof reaching a given set time to be an extreme value; and restarting the procedure and determining a next extreme value includes:
receiving the physiological signal value, starting a counter a and adding an unit value to the counter a, comparing the physiological signal value with the register value; if the physiological signal value is greater than/less than the register value, replacing the register value with the physiological signal value, adding a count value of the counter a to an accumulator b, and clearing the counter a to zero; if the physiological signal value is less than/greater than the register value, keeping receiving a next heartbeat signal voltage; determining the corresponding physiological signal value to be an extreme value when the count value of the counter a reaches a value corresponding to the give set time, outputting a value accumulated in the accumulator b, clearing the counter, a register, and the accumulator b, and keeping determining the next extreme value; the extreme value and the next extreme value being respectively a maximum value or a minimum value.

Preferably, the step of obtaining the cycle of the physiological signal by calculating a time difference between the extreme value and the next extreme value includes:
obtaining the cycle of the physiological signal by adding a time required for reaching the value accumulated in the accumulator b and the given set time.

Preferably, the given set time is longer than a half cycle of an upper limit of a recognition range of the cycle of the physiological signal.

The present disclosure further provides a system for obtaining a cycle of a physiological signal, including:
an extreme value judging unit, configured for receiving a physiological signal value and a register value, comparing the physiological signal value with the register value, and reserving one of the physiological signal value and the register value; determining the physiological signal value with a time duration thereof reaching a given set time to be an extreme value; and restarting the procedure and determining a next extreme value;
a cycle calculating unit, configured for obtaining a cycle of the physiological signal by calculating a time difference between the extreme value and the next extreme value.

Preferably, the system further includes:
a close cycle judging unit, configured for judging whether two cycles respectively obtained in a maximum value way and in a minimum value way in a time duration are close to each other; and
a cycle determining unit, configured for determining an average of the two cycles to be the cycle of the physiological signal if the two cycles are close to each other.

Preferably, the extreme value judging unit is configured for: receiving the physiological signal value, starting a counter a and adding an unit value to the counter a, comparing the physiological signal value with the register value; if the physiological signal value is greater than/less than the register value, replacing the register value with the physiological signal value, adding a count value of the counter a to an accumulator b, and clearing the counter a to zero; if the physiological signal value is less than/greater than the register value, keeping receiving a next heartbeat signal voltage; determining the corresponding physiological signal value to be an extreme value when the count value of the counter a reaches a value corresponding to the give set time, outputting a value accumulated in the accumulator b, clearing the counter a, a register, and the accumulator b, and keeping determining the next extreme value; the extreme value and the next extreme value being respectively a maximum value or a minimum value.

Preferably, the cycle calculating unit is configured for: obtaining the cycle of the physiological signal by adding a time required for reaching the value accumulated in the accumulator and the given set time.

Preferably, the given set time is longer than a half cycle of an upper limit of a recognition range of the cycle of the physiological signal.

In the present disclosure, the cycle of the physiological signal can be obtained by the extreme value recognition algorithm, which is simple, fast, of high efficiency, and of high reliability; furthermore, the requirements about the amplification of the physiological signal, the filtering of the physiological signal, and the analogy-to-digital conversion are relatively low and the data process is relatively simple, thus, the hardware cost can be greatly reduced.

### DESCRIPTION OF THE DRAWINGS

Many aspects of the embodiments can be better understood with reference to the following drawings. The components in the drawings are not necessarily dawns to scale, the emphasis instead being placed upon clearly illustrating the principles of the embodiments. Moreover, in the drawings, like reference numerals designate corresponding parts throughout the several views.

FIG. 1 is a schematic view of a waveform of a signal obtained by a piezoelectric sensor in a related art;

FIG. 2 is a schematic view of a waveform of a bioelectric signal in the related art;

FIG. 3 is a flow chart of a method for obtaining a cycle of a physiological signal in accordance with an embodiment of the present disclosure;

FIG. 4 is a flow chart of a method for obtaining a cycle of a physiological signal in accordance with another embodiment of the present disclosure;

FIG. 5 is a schematic view of a system for obtaining a cycle of a physiological signal in accordance with an embodiment of the present disclosure; and

FIG. 6 is a schematic view of a system for obtaining a cycle of a physiological signal in accordance with another embodiment of the present disclosure.

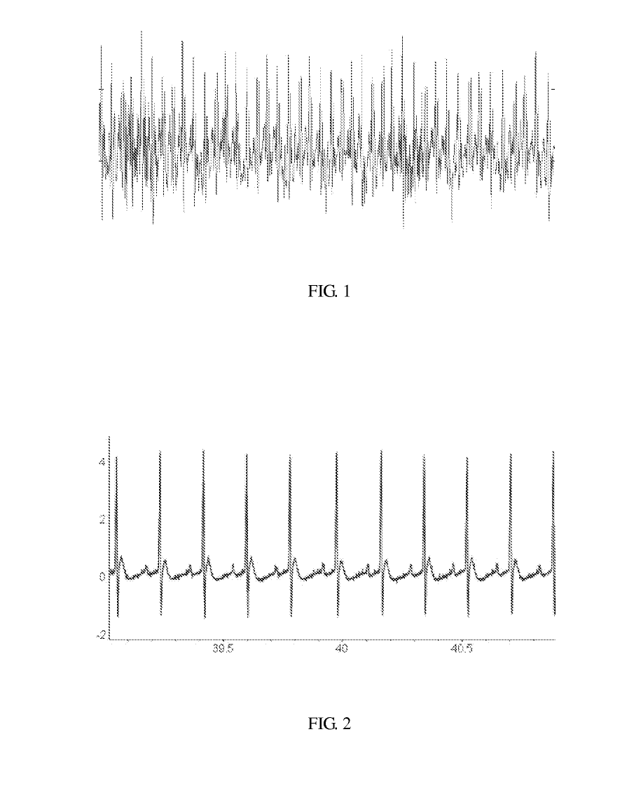

### DETAILED DESCRIPTION

The disclosure is illustrated by way of example and not by way of limitation in the figures of the accompanying drawings in which like references indicate similar elements. It should be noted that references to “an” or “one” embodiment is this disclosure are not necessarily to the same embodiment, and such references mean at least one.

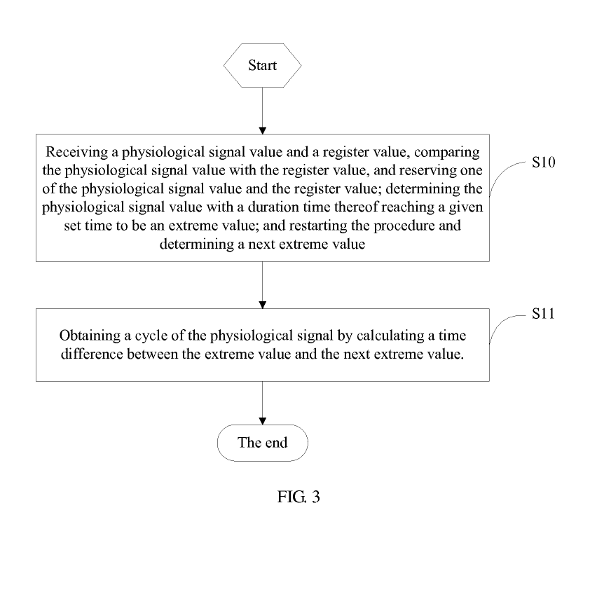

Referring to FIG. 3, a method for obtaining a cycle of a physiological signal in accordance with an embodiment of the present disclosure is provided. The method includes:
step S10, receiving a physiological signal value and a register value, comparing the physiological signal value with the register value, and reserving one of the physiological signal value and the register value; determining the physiological signal value with a duration time thereof reaching a given set time to be an extreme value; and restarting the procedure and determining a next extreme value; and
step S11, obtaining a cycle of the physiological signal by calculating a time difference between the extreme value and the next extreme value.

In the embodiment, the physiological signal can be a respiration signal or a heartbeat signal, the physiological signal value can be a specific value such as a voltage, and the physiological signal can be obtained by a piezoelectric sensor. In the above method for obtaining the cycle of the physiological signal, a micro-motion sensor such as the piezoelectric sensor can be used for obtaining a micro-motion signal of a human body when the human body is in a quiet state, and the cycle of the physiological signal such as the respiration signal and/or the heartbeat signal can be directly determined in a carrier signal.

The step S10 specifically includes: receiving one physiological signal value, starting a counter a and adding an unit value to the counter a, and comparing the physiological signal value with the register value; if the physiological signal value is greater than/less than the register value, replacing the register value with the physiological signal value, adding a count value of the counter a to an accumulator b, and clearing the counter a to zero; if the physiological signal value is less than/greater than the register value, keeping receiving a next voltage of the physiological signal; when the count value of the counter a reaches a value corresponding to the given set time, determining the physiological signal value to be an extreme value, outputting the value accumulated in the accumulator b, and clearing the counter a, a register, and the accumulator b, and continuously determining a next extreme value. The register value can be equal to zero or the reserved physiological signal value.

The step S11 can specifically include: obtaining the cycle of the physiological signal by adding the time required for reaching the value accumulated in the accumulator b and the given set time.

The cycle of the physiological signal can be obtained by an extreme value recognition algorithm. The extreme value can be a maximum value or a minimum value, that is, the current extreme value and the next extreme value can respectively be a maximum value or a minimum value. The maximum value recognition algorithm can be: timing a duration when one received physiological signal value is kept greatest, and determining the received physiological signal value to be a maximum value in one cycle when the timer reaches the given set time. The time difference (time length) between the maximum value (the extreme value) and a next maximum value (the next extreme value) can be calculated. The minimum value recognition algorithm can be: timing a duration when one received physiological signal is kept smallest, and determining the received physiological signal value to be a minimum value in one cycle when the timer reaches the given set time. Furthermore, the time difference between the minimum value (the extreme value) and a next minimum value (the next extreme value) can be calculated. The cycle of the physiological signal obtained by the maximum value recognition algorithm and the cycle of the physiological signal obtained by the minimum value recognition algorithm are respectively obtained according to the given set time and the time difference between the two maximum values or the time difference between the two minimum values. The given set time can be set according to the specific physiological signal, for example, in the embodiment the given set time can be longer than a half of the cycle of the physiological signal.

The method for obtaining the above physiological signal can further be described as follows based on the following example in which the cycle of the physiological signal is obtained in the maximum value way.

At first, receiving a voltage of a heartbeat signal outputted from the piezoelectric sensor, starting the counter a and adding 1 to the counter a, comparing the count value of the counter a with the register value in the register; if the count value is greater than the register value, replacing the register value with the count value, adding the count value of the counter a to the accumulator b, and clearing the counter a to zero; if the count value is less than the register value; keeping receiving the next voltage of the heartbeat signal.

Outputting the value accumulated in the accumulator b, clearing the counter a, the register, and the accumulator b, and starting a new cycle when the count value of the counter a reaches a corresponding value. Each heartbeat signal received in the new cycle is compared with the register value 0 in the register. The formula for calculating the cycle of the above heartbeat signal is expressed as:

Cycle (s)=(the value accumulated in the accumulator b/a receiving speed of the physiological signal)+the given set time.

Since the constant of the time duration (that is, the given set time) when the physiological signal is kept being greatest needs to be previously set, thus, an upper limit of the recognition cycle can be the reciprocal of the given set time.

For example, if the frequency of the heartbeat signal ranges from 0.7 Hz to 1.6 Hz, then the given set time of the maximum value is 0.55 seconds (the given set time of the minimum value is 0.6 seconds). If a received heartbeat signal is kept being greatest in the register for 0.55 seconds, then the counter a starts to count. Adding 1 to the counter a when a new heartbeat signal is inputted into the comparator. If the voltage of the new heartbeat signal is greater than the previous register value, storing the voltage of the new heartbeat signal in the register and replacing the previous register value with the voltage, adding the count value of the counter a to the accumulator b, and clearing the counter to zero. If the voltage of the new heartbeat signal is less than the previous register value stored in the register, adding 1 to the counter, continuing the cycle of comparing the voltage of the heartbeat signal, outputting a value B accumulated in the accumulator b until the count value of the counter a is equal to the number of the voltages (for example, 300) of the heartbeat signals received in 0.55 seconds, and adding 0.55 seconds and the time required for reaching the value B accumulated in the accumulator b to acquire the cycle of the heartbeat signal.

Supposed that the number of the received heartbeat signals counted by the counter a in 0.55 seconds when one heartbeat signal is kept being greatest is 275, and the speed of receiving the heartbeat signal is 500 per second, then the cycle of the heartbeat signal=(B/500)+0.55.

According to the above method, the cycle of the physiological signal is respectively obtained in the maximum value way and in the minimum way in a time duration, thereafter the more accurate cycle of the physiological signal can be obtained according to the cycle of the physiological signal obtained in the maximum value way and the cycle of the physiological signal obtained in the minimum value way.

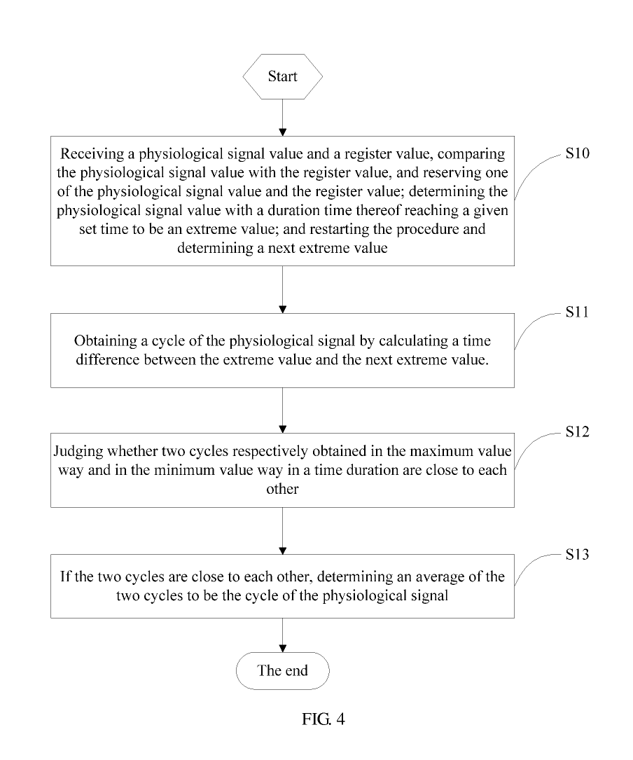

Referring to FIG. 4, in another embodiment of the present disclosure, the method for obtaining the cycle of the physiological signal described above can further include the following steps:
step S12, judging whether two cycles respectively obtained in the maximum value way and in the minimum value way in a time duration are close to each other; and
step S13, if the two cycles are close to each other, determining an average of the two cycles to be the cycle of the physiological signal.

In a given set time duration, for example, at least one cycle of the physiological signal obtained in the maximum value way and at least one cycle of the physiological signal obtained in the minimum value way are obtained. The cycles obtained in the two ways are compared with each other to judge whether the two cycles are close to each other. If the two cycles are close to each other, averaging the two cycles and determining the average of the two cycles to be the more accurate cycle of the physiological signal, otherwise ending the procedure. Whether the two cycles are close to each other or not can be judged according to the specific type of the physiological signal, for example, the time difference between two close cycles of a respiration signal is approximately 0.004 seconds and the time difference between two close cycles of a heartbeat signal is approximately 0.017 seconds.

In the above method for obtaining the cycle of the physiological signal, the cycle of the physiological signal can be obtained by the extreme value recognition algorithm, which is simple, fast, of high efficiency, and of high reliability; furthermore, the requirements about the amplification of the physiological signal, the filtering of the physiological signal, and the analogy-to-digital conversion are relatively low and the data process is relatively simple, thus, the hardware cost can be greatly reduced.

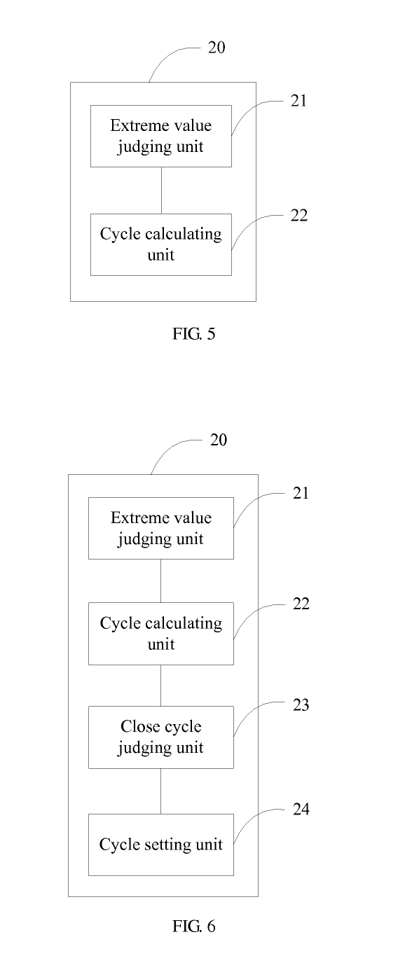

Referring to FIG. 5, a system 20 for obtaining a cycle of a physiological signal in accordance with an embodiment of the present disclosure is provided. The system 20 includes an extreme value judging unit 21 and a cycle calculating unit 22. The extreme value judging unit 21 is configured for receiving a physiological signal value and a register value, comparing the physiological signal value with the register value, and reserving one of the physiological signal value and the register value; determining the physiological signal value with a time duration thereof reaching a given set time to be an extreme value; and restarting the procedure and determining a next extreme value. The cycle calculating unit 22 is configured for obtaining a cycle of the physiological signal by calculating a time difference between the extreme value and the next extreme value.

In the embodiment, the physiological signal can be a respiration signal or a heartbeat signal, the physiological signal value can be a specific value such as a voltage, and the physiological signal can be obtained by a piezoelectric sensor. In the above system for obtaining the cycle of the physiological signal, a micro-motion sensor such as the piezoelectric sensor can be used for obtaining a micro-motion signal of a human body when the human body is in a quiet state, and the cycle of the physiological signal such as the respiration signal and/or the heartbeat signal can be directly determined in a carrier signal.

The extreme value judging unit 21 is specifically used for: receiving one physiological signal value, starting a counter a and adding an unit value to the counter, and comparing the physiological signal value with the register value; if the physiological signal value is greater than/less than the register value, replacing the register value with the physiological signal value, adding a count value of the counter to an accumulator b, and clearing the counter to zero; if the physiological signal value is less than/greater than the register value, keeping receiving a next voltage of the heartbeat signal; when the count value of the counter reaches a value corresponding to the given set time, determining the physiological signal value to be an extreme value, outputting a value accumulated in the accumulator b, and clearing the counter a, a register, and the accumulator b, and continuously determining the next extreme value. The register value can be equal to zero or the reserved physiological signal value.

The cycle calculating unit 22 is specifically used for: obtaining the cycle of the physiological signal by adding a time required for reaching the value accumulated in the accumulator b and the given set time.

The cycle of the physiological signal can be obtained by an extreme value recognition algorithm. The extreme value can be a maximum value or a minimum value, that is, the current extreme value and the next extreme value can respectively be a maximum value or a minimum value. The maximum value recognition algorithm can be: timing a duration when one received physiological signal value is kept greatest, and determining the received physiological signal value to be a maximum value in one cycle when the timer reaches the given set time. The time difference (time length) between the maximum value (the extreme value) and a next maximum value (the next extreme value) can be calculated. The minimum value recognition algorithm can be: timing a duration when one received physiological signal is kept smallest, and determining the received physiological signal value to be a minimum value in one cycle when the timer reaches the given set time. Furthermore, the time difference between the minimum value (the extreme value) and a next minimum value (the next extreme value) can be calculated. The cycle of the physiological signal obtained by the maximum value recognition algorithm and the cycle of the physiological signal obtained by the minimum value recognition algorithm are respectively obtained according to the given set time and the time difference between two maximum values or the time difference between two minimum values. The given set time can be set according to the specific physiological signal, for example, in the embodiment the given set time can be longer than a half of the cycle of the physiological signal.

The system 20 for obtaining the above physiological signal can further be described as follows based on the following example in which the cycle of the physiological signal is obtained in the maximum value way.

At first, receiving a voltage of a heartbeat signal outputted from the piezoelectric sensor, starting the counter a and adding 1 to the counter a, comparing the count value of the counter a with the register value in the register; if the count value is greater than the register value, replacing the register value with the count value, adding the count value of the counter a to the accumulator b, and clearing the counter a to zero; if the count value is less than the register value; keeping receiving the next voltage of the heartbeat signal.

Outputting the value accumulated in the accumulator b, clearing the counter a, the register, and the accumulator b, and starting a new cycle when the count value of the counter a reaches a corresponding value. Each heartbeat signal received in the new cycle is compared with the register value 0 in the register. The formula for calculating the cycle of the above heartbeat signal is expressed as:

Cycle (s)=(the value accumulated in the accumulator b/a receiving speed of the physiological signal)+the given set time.

Since the constant of the time duration (that is, the given set time) when the physiological signal is kept being greatest needs to be previously set, thus, an upper limit of the recognition cycle can be the reciprocal of the given set time.

For example, if the frequency of the heartbeat signal ranges from 0.7 Hz to 1.6 Hz, then the given set time of the maximum value is 0.55 seconds (the given set time of the minimum value is 0.6 seconds). If a received heartbeat signal is kept being greatest in the register for 0.55 seconds, then the counter a starts to count. Adding 1 to the counter a when a new heartbeat signal is inputted into the comparator. If the voltage of the new heartbeat signal is greater than the previous register value, storing the voltage of the new heartbeat signal in the register and replacing the previous register value with the voltage, adding the count value of the counter a to the accumulator b, and clearing the counter to zero. If the voltage of the new heartbeat signal is less than the previous register value stored in the register, adding 1 to the counter, continuing the cycle of comparing the voltage of the heartbeat signal, outputting a value B accumulated in the accumulator b until the count value of the counter a is equal to the number of the voltages (for example, 300) of the heartbeat signals received in 0.55 seconds, and adding 0.55 seconds and the time required for reaching the value B in the accumulator b to acquire the cycle of the heartbeat signal.

Supposed that the number of the received heartbeat signals counted by the counter a in 0.55 seconds when one received heartbeat signal is kept being greatest is 275, and the speed of receiving the heartbeat signal is 500 per second, then the cycle of the heartbeat signal=(B/500)+0.55.

According to the above method, the cycle of the physiological signal is respectively obtained in the maximum value way and in the minimum value way in a time duration, thereafter the more accurate cycle of the physiological signal can be obtained according to the cycle of the physiological signal obtained in the maximum value way and the cycle of the physiological signal obtained in the minimum value way.

Referring to FIG. 6, in another embodiment of the present disclosure, the system can further include a close cycle judging unit 23 and a cycle setting unit 24. The close cycle judging unit 23 is used for judging whether two cycles respectively obtained in the maximum value way and in the minimum value way in a time duration are close to each other. The cycle setting unit 24 is used for setting an average of the two cycles to be the cycle of the physiological signal if the two cycles are close to each other.

In a given set time duration, at least one cycle of the physiological signal obtained in the maximum value way and at least one cycle of the physiological signal obtained in the minimum value way are obtained. The two cycles obtained in the two ways are compared with each other to judge whether the two cycles are close to each other. If the two cycles are close to each other, averaging the two cycles and determining the average of the two cycles to be the more accurate cycle of the physiological signal, otherwise ending the procedure. Whether the two cycles are close to each other or not can be judged according to the specific type of the physiological signal, for example, the time difference between two close cycles of a respiration signal is approximately 0.004 seconds and the time difference between two close cycles of a heartbeat signal is approximately 0.017 seconds.

In the above system 20 for obtaining the cycle of the physiological signal, the cycle of the physiological signal can be obtained by the extreme value recognition algorithm, which is simple, fast, of high efficiency, and of high reliability; furthermore, the requirements about the amplification of the physiological signal, the filtering of the physiological signal, and the analogy-to-digital conversion are relatively low and the data process is relatively simple, thus, the hardware cost can be greatly reduced.

Even though information and the advantages of the present embodiments have been set forth in the foregoing description, together with details of the mechanisms and functions of the present embodiments, the disclosure is illustrative only; and that changes may be made in detail, especially in matters of shape, size, and arrangement of parts within the principles of the present embodiments to the full extend indicated by the broad general meaning of the terms in which the appended claims are expressed.

1. A method for obtaining a cycle of a physiological signal, comprising:
receiving a physiological signal value and a register value, comparing the physiological signal value with the register value, and reserving one of the physiological signal value and the register value; determining the physiological signal value with a time duration thereof reaching a given set time to be an extreme value; and restarting the procedure and determining a next extreme value; and
obtaining a cycle of the physiological signal by calculating a time difference between the extreme value and the next extreme value.

2. The method of claim 1 further comprising the following steps:
judging whether two cycles respectively obtained in a maximum value way and in a minimum value way in a time duration are close to each other; and
determining an average of the two cycles to be the cycle of the physiological signal if the two cycles are close to each other.

3. The method of claim 1, wherein the step of receiving a physiological signal value and register value and comparing the physiological signal value with the register value; determining the physiological signal value with a time duration thereof reaching a given set time to be an extreme value; and restarting the procedure and determining a next extreme value comprises:
receiving the physiological signal value, starting a counter a and adding an unit value to the counter a, comparing the physiological signal value with the register value; if the physiological signal value is greater than/less than the register value, replacing the register value with the physiological signal value, adding a count value of the counter a to an accumulator b, and clearing the counter a to zero; if the physiological signal value is less than/greater than the register value, keeping receiving a next physiological signal value; determining the corresponding physiological signal value to be an extreme value when the count value of the counter a reaches a value corresponding to the give set time, outputting a value accumulated in the accumulator b, clearing the counter a, a register, and the accumulator b, and keeping determining the next extreme value; the extreme value and the next extreme value being respectively a maximum value or a minimum value.

4. The method of claim 3, wherein the step of obtaining a cycle of the physiological signal by calculating a time difference between the extreme value and the next extreme value comprises:
obtaining the cycle of the physiological signal by adding a time required for reaching the value accumulated in the accumulator b and the given set time.

5. The method of claim 1, wherein the given set time is longer than a half cycle of an upper limit of a recognition range of the cycle of the physiological signal.

6. A system for obtaining a cycle of a physiological signal, comprising:
an extreme value judging unit, configured for receiving a physiological signal value and a register value, comparing the physiological signal value with the register value, and reserving one of the physiological signal value and the register value; determining the physiological signal value with a time duration thereof reaching a given set time to be an extreme value; and restarting the procedure and determining a next extreme value;
a cycle calculating unit, configured for obtaining a cycle of the physiological signal by calculating a time difference between the extreme value and the next extreme value.

7. The system of claim 6 further comprising:
a close cycle judging unit, configured for judging whether two cycles respectively obtained in a maximum value way and in a minimum value way in a time duration are close to each other; and
a cycle determining unit, configured for determining an average of the two cycles to be the cycle of the physiological signal if the two cycles are close to each other.

8. The system of claim 6, wherein the extreme value judging unit is configured for: receiving the physiological signal value, starting a counter a and adding an unit value to the counter a, comparing the physiological signal value with the register value; if the physiological signal value is greater than/less than the register value, replacing the register value with the physiological signal value, adding a count value of the counter a to an accumulator b, and clearing the counter a to zero; if the physiological signal value is less than/greater than the register value, keeping receiving a next physiological signal value; determining the corresponding physiological signal value to be an extreme value when the count value of the counter a reaches a value corresponding to the give set time, outputting a value accumulated in the accumulator b, clearing the counter a, a register, and the accumulator b, and keeping determining the next extreme value; the extreme value and the next extreme value being respectively a maximum value or a minimum value.

9. The system of claim 6, wherein the cycle calculating unit is configured for: obtaining the cycle of the physiological signal by adding a time required for reaching the value accumulated in the accumulator and the given set time.

10. The system of claim 6, wherein the given set time is longer than a half cycle of an upper limit of a recognition range of the cycle of the physiological signal.

11. The method of claim 2, wherein the given set time is longer than a half cycle of an upper limit of a recognition range of the cycle of the physiological signal.

12. The method of claim 3, wherein the given set time is longer than a half cycle of an upper limit of a recognition range of the cycle of the physiological signal.

13. The system of claim 7, wherein the given set time is longer than a half cycle of an upper limit of a recognition range of the cycle of the physiological signal.

14. The system of claim 8, wherein the given set time is longer than a half cycle of an upper limit of a recognition range of the cycle of the physiological signal.
